(19) United States
(12) Patent Application Publication
(10) Pub. No.: US 2005/0090783 A1
(43) Pub. Date: Apr. 28, 2005

(54) Colorful shielded reciprocating butterfly needle

(76) Inventor: Wilmer L. Sibbitt JR., Albuquerque, NM (US)

Correspondence Address: JAGTIANI + GUTTAG, 10363-A DEMOCRACY LANE, FAIRFAX, VA, 22030, US

(21) Appl. No.: 10/693,082

(22) Filed: Oct. 27, 2003

(51) Int. Cl. A61M 5/00

(52) U.S. Cl. 604/263

(57) ABSTRACT

A butterfly needle assembly has a needle and a shield with wings integral to either the needle or the shield. The wings have aesthetically pleasing patterns to distract the patient during treatment. The mechanical design of the needle and shield juncture improves the stability of the assembly when inserted into a patient, as well as allowing the assembly to be disengaged with a single hand to help the caregiver avoid a needle “stick” and prevent the spread of diseases.

### BACKGROUND OF THE INVENTION

1. Field of the Invention

The present invention relates generally to butterfly needles or catheters, and more particularly to unintimidating, safer butterfly needles capable of single-handed manipulation. These butterfly needles are aesthetically pleasing while reducing the probability of accidental needle sticks and providing health care professionals with a free hand to comfort or assist the patient.

2. Description of the Prior Art

Butterfly needles consist of 1) a needle or catheter, 2) a plastic hub, 3) wings attached to the side of the hub, and 4) a catheter or fitting lumen attached to the hub contiguously and continuously with the lumen of the needle or catheter. Butterfly needles are popular in pediatric medicine as well as for use with small or fragile veins in adults. A traditional butterfly needle is unsheathed or uncapped, with the wings grasped as a handle, to penetrate the skin, and then the butterfly wings are folded down and taped to the skin. Despite their popularity, traditional butterfly needles present a number of problems.

From a young age, children fear needles as they associate the pain of their immunizations with the administering needle. Butterfly needles are commonly used in situations when the patient is facing an occasion more traumatic than a simple shot. Often patients, including children, fear their treatment and face feelings of despair. It would be encouraging if the needle, vital to their treatment, did not intimidate or frighten them further.

Conventional butterfly needles are very dangerous after use and can easily result in a needle stick. Accidental penetration of the skin from sharp instruments is one of the most common modes of transmission of fatal or debilitating infectious diseases to health care workers. Hepatitis B, hepatitis C, and HIV (the AIDS virus) in the health care environment are typically transmitted from needle sticks and result in years of debilitating illness, loss of productivity, workman's compensation payments, medical expenses, and accelerated mortality. A partial solution to this problem has been the use of guarded needles and syringes.

A major disadvantage to conventional shielding solutions is that almost all contemporary devices require two hands to inactivate the needle. Generally the shielding device is held with one hand and the catheter, which is attached to the needle, is pulled to bring the needle into the shielding device where it is then inactivated. This requirement for two hands to inactivate intravenous catheters is a major disadvantage, as it prevents one hand of the operator from applying pressure to a puncture site after removing a needle. This is particularly true in children, squeamish patients, or very ill patients who cannot apply pressure themselves. In this situation, there is an exposed and contaminated needle capable of contaminating the environment or inadvertently sticking the operator while applying pressure to the puncture site. This general requirement for two-handed inactivation is a characteristic of all contemporary shielded butterfly needles.

Another major problem with many traditional butterfly needles, especially those with a rigid shield, is that the shield makes the butterfly device effectively longer, creating a longer lever arm. With a longer effective device, slight changes in orientation can cause major changes in the position of the needle tip in relation to the fulcrum of the device causing disruption of the blood vessel or painful tension on the tissues. This longer lever arm especially becomes a problem when the device is taped to the skin or manipulated.

The final step in stabilizing any butterfly needle is the folding down of the plastic wings onto skin and fixing them onto the skin with medical adhesive tape. However, there are moments of instability while the operator is holding down the butterfly needle with one hand, and reaching for a piece of tape with the other. In this moment, the butterfly needle may become dislodged, abrogating the entire procedure.

### SUMMARY OF THE INVENTION

It is therefore an object of the present invention to provide a butterfly needle that will aesthetically comfort and soothe a patient.

It is a further object to provide a butterfly needle with an easy, safe method of one-handed inactivation.

It is yet another object of the invention to provide a shielded butterfly needle free of the deleterious effects of a longer level arm.

Finally, it is an object of the invention to provide a butterfly needle easily fixable to the skin while permitting greater control of the needle.

According to a first broad aspect of the present invention, there is provided a butterfly needle assembly comprising a needle having a needle hub, a locking means integral with the needle hub having a tab protruding radially from the needle hub, and a shield with a distal end and a proximal end having integral wings and a dorsal track extending axially along the shield wherein the tab extends through the dorsal track such that when in operation as the needle moves through the shield, the tab travels along the dorsal track to engage a cut-out at the distal end of the dorsal track thereby locking the needle within the shield.

According to second broad aspect of the invention, there is provided a butterfly needle comprising a needle having a needle hub wherein the needle hub has a pair of wings extending therefrom and the wings are aesthetically decorated.

According to a third broad aspect of the present invention, there is provided a butterfly needle assembly comprising a needle having a needle hub with integral wings extending radially from the needle hub, a locking means integral with the needle hub having a tab protruding radially from the needle hub in a plane perpendicular to the integral wings, and a shield with a distal end and a proximal end and a dorsal track extending axially along the shield and the shield further having side tracks wherein the tab extends through the dorsal track and the integral wings extend through the side tracks such that when in operation as the needle moves through the shield, the tab travels along the dorsal track to engage a cut-out at the distal end of the dorsal track thereby locking the needle within the shield.

Other objects and features of the present invention will be apparent from the following detailed description of the preferred embodiment.

### BRIEF DESCRIPTION OF THE DRAWINGS

The invention will be described in conjunction with the accompanying drawings, in which:

FIG. 1 is a conventional butterfly needle;

FIGS. 2A, 2B, 2C and 2D illustrate alternative wing structures to present an aesthetically pleasing butterfly needle in accordance with a preferred embodiment of the present invention;

FIGS. 3A, 3B, 3C and 3D show alternative placements for a wing along the lumen of a catheter;

FIGS. 4A, 4B, 4C and 4D illustrate alternative natural markings for the wings of the butterfly needle in accordance with a preferred embodiment of the present invention;

FIGS. 5A, 5B, 5C, 5D, 5E, 5F, 5G and 5H are views of alternative needles that are non-butterfly shapes and designs, and FIG. 5I shows a coordinating bandage;

FIG. 6 illustrates fold-out adhesive tape on the back of the wings to anchor the needle to the skin;

FIG. 7 shows the option of an adhesive butterfly needle wherein adhesive or pressure sensitive adhesive foam is on the skin-side of the butterfly wings;

FIGS. 8A and 8B show the components of a winged butterfly shield with a dorsal track and thumb rest;

FIGS. 9A and 9B show multiple views of the assembled butterfly shield of FIG. 8 in extended and retracted positions in accordance with a preferred embodiment of the present invention;

FIGS. 10A, 10B, 10C, 10D, 10E and 10F illustrate multiple mechanical devices for the locking mechanism of the needle and shield;

FIGS. 11A and 11B show a butterfly needle with dorsal and side tracks on the needle rather than the shield as shown in FIG. 8;

FIGS. 12A and 12B show multiple views of the assembled needle of FIG. 11 in extended and retracted positions in accordance with a preferred embodiment of the present invention;

FIGS. 13A and 13B illustrate the components of an arched shielded butterfly needle in accordance with a preferred embodiment of the present invention;

FIGS. 14A and 14B show the assembled arched shielded butterfly needle of FIG. 13;

FIGS. 15A, 15B, 15C, 15D, 15E and 15F show assembled variants of a reciprocating butterfly needle in accordance with a preferred embodiment of the present invention; and

FIGS. 16A, 16B, 16C, 16D and 16E present alternative slot formations and slot locking mechanisms, and FIGS. 16F, 16G, 16H, 16I, 16J and 16K suggest bendable shields in extended and flexed positions.

### DETAILED DESCRIPTION OF THE PREFERRED EMBODIMENT

It is advantageous to define several terms before describing the invention. It should be appreciated that the following definitions are used throughout this application.

### Definitions

Where the definition of terms departs from the commonly used meaning of the term, applicant intends to utilize the definitions provided below, unless specifically indicated.

For the purposes of the present invention, “aesthetically decorated” refers to any decoration that creates a more aesthetically pleasing appearance on the wing structure.

For the purposes of the present invention, “animal” refers to any aquatic, terrestrial, or flying animal, whether real or fictional.

For the purposes of the present invention, “cartoon character” refers to a unique, trademarked or copyrighted pictorial representation or caricature.

For the purposes of the present invention, “catheter” refers to a tubular metal or rubber that forms a passageway to a needle.

For the purposes of the present invention, “continuously and contiguously connected” refers to the joint of the hub and the lumen of the needle or catheter wherein a continuous seam exists at the joint thereby allowing matter within the lumen to flow uninterrupted into the hub.

For the purposes of the present invention, “cut-out” refers to a recess at an endpoint of a track to engage a device to lock the needle in place.

For the purposes of the present invention, “dentate” refers to having teeth or tooth-like projections or notches.

For the purposes of the present invention, “dorsal track” refers to a path in a shield allowing a locking device to travel axially along the shield.

For the purposes of the present invention, “driveline” refers to any internal mechanism communicating motion to the needle from a plunger or tab.

For the purposes of the present invention, “drive tab” refers to a tab that is moved within the track and as a result of transference, moves the needle.

For the purposes of the present invention, “fitting” refers to a lead portion of a catheter by which it may be connected to other components of the assembly.

For the purposes of the present invention, “hilt” refers to the handle or portion by which the needle may be held.

For the purposes of the present invention, “holiday novelty” refers to a symbol or representation of a holiday.

For the purposes of the present invention, “hub” refers to a plastic attachment at the back of the needle by which the needle may be connected to other components of the assembly.

For the purposes of the present invention, “integral” refers to the characteristic of two components being attached to each other in a manner to inhibit separation, such as by adhesive, molding, etc.

For the purposes of the present invention, “irreversibly” refers to a status whereby the needle may not be disengaged from the track.

For the purposes of the present invention, “locking device” refers to a mechanical connection whereby movement is inhibited by the connection of two components.

For the purposes of the present invention, “lumen” refers to a passageway for connection between a needle and/or a catheter.

For the purposes of the present invention, “needle assembly” refers to a copulation of a needle or catheter, wings, and a shield.

For the purposes of the present invention, “pinching motion” refers to a motion similar to squeezing between a finger and the thumb.

For the purposes of the present invention, “prehistoric creature” refers to a living being from a period antecedent to the earliest period of recorded history.

For the purposes of the present invention, “shield” refers to a plastic tubular channel way to enclose the joint between a needle hub and the lumen. Wings may extend from or through the shield.

For the purposes of the present invention, “stop” refers to a protrusion that inhibits motion.

For the purposes of the present invention, “thumb rest” refers to an aperture on the shield on which an operator may set his thumb.

For the purposes of the present invention, “tracks” refer to open paths in the shield that allow movement therethrough.

For the purposes of the present invention, “tubing” refers to material in the form of a tube.

For the purposes of the present invention, “wings” refer to dual radial extensions from a shield or needle hub.

### Description

A traditional butterfly needle consists of 1) a needle or catheter, 2) a plastic hub, 3) wings attached to the side of the hub, and 4) a catheter or fitting for a catheter attached to the hub which is contiguously and continuously connected with the lumen of the needle or catheter. Shielded variants of these needles also exist with wings attached to the needle assembly, as in a traditional butterfly needle, or attached to the shield. There are however major problems with all contemporary designs of butterfly needles. The present invention addresses the psychological, aesthetic, safety, ergonomic, and stability problems of traditional and shielded butterfly needles and butterfly catheters. The individual solutions and principals to rectify these problems constitute the present invention. As will be apparent to those skilled in the art, this invention may also be applied to non-winged needles and catheters.

The present invention includes both conventional and shielded butterfly needles and catheters with specific modifications of the wings and shield to make these devices less threatening, more interesting, and more distracting from the painful task at hand, while at the same time involving the patient directly in their own medical care. These modifications consist of specific and general color patterns of the wings to attract and distract the patient's attention, changes in the design and shape of the wings to achieve new and exciting aesthetic effects, and modifications of the taping systems to enhance and amplify the aesthetic improvements and stabilize the needle, while at the same time providing the patient with a choice of different butterfly needle patterns and colors. These inventions permit the patients to make a choice of their own aesthetically pleasing butterfly needle or catheter and, thus, the patients will become directly and voluntarily involved in their own medical care. These modifications are especially useful in pediatric medicine, but are also of interest to beneficially distract adult patients. The devices of the present invention also indicate to patients of all ages that the nurses, technicians, and physicians care about patients'feelings. Use of these interesting and aesthetically pleasing devices coupled with patient choice make a bad experience better, gentler, kinder, more interesting, and more meaningful.

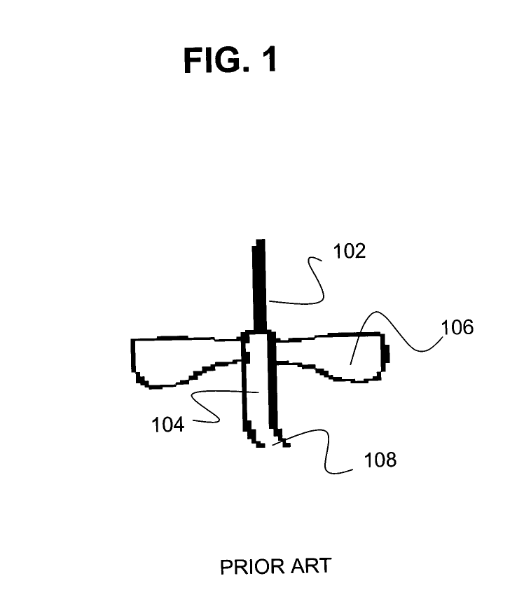

The plastic that is used for a butterfly needle, whether shielded or non-shielded, is usually monotone and generally of a darker hue, which does not distract the patient from the painful procedure, but rather makes the butterfly needle look like a cold, hard, medical device. A typical butterfly needle is shown in FIG. 1. The needle 102 and wings 106 are designed to be functional connected to tubing 108 by the lumen 104, with no attention in the design to the psychological impact upon the patient when these needles are inserted.

The present invention diminishes the negative design effect of traditional butterfly needles by dispensing with dark monotones and making the wings interesting and attractive with the use of bright colors, hues, reflecting surfaces, patterns, and designs to make the needle more interesting while distracting the patient. Although the examples of the present invention are illustrated in black and white, it is contemplated by the present invention that embodiments of the present invention may have variable bright colors, metallic and reflective surfaces, glittery surfaces, appear transparent or translucent, or have dramatic surface designs.

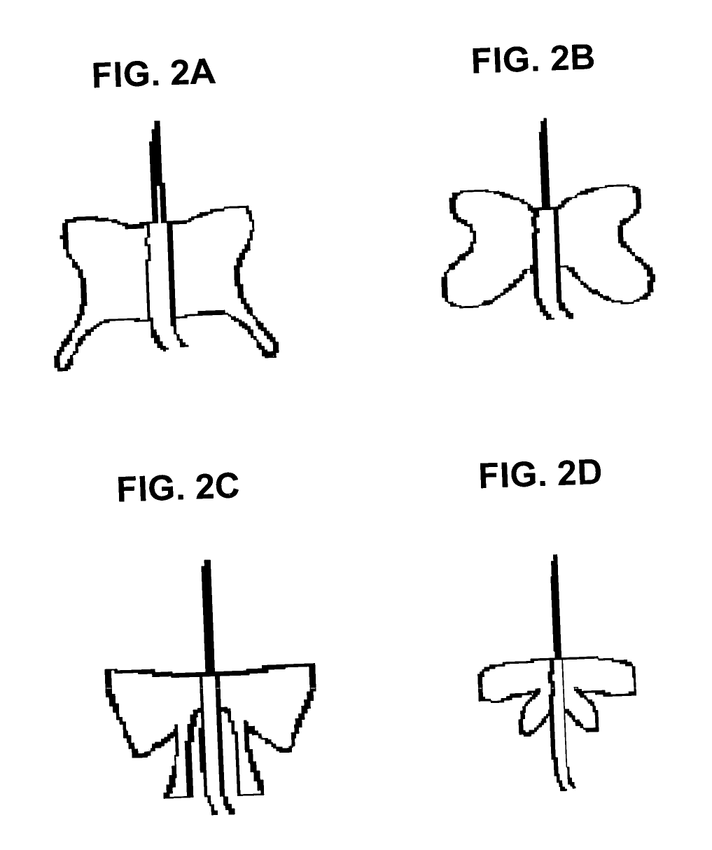

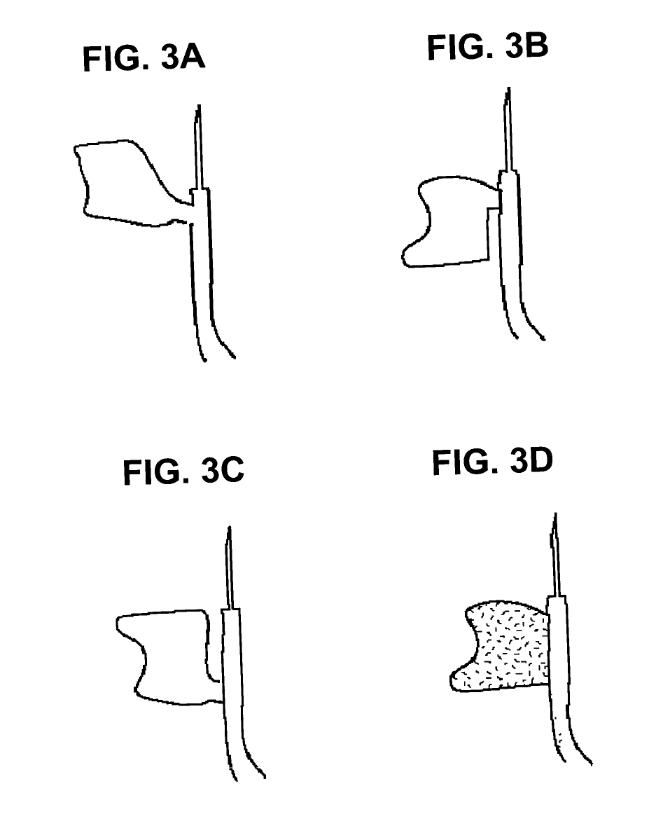

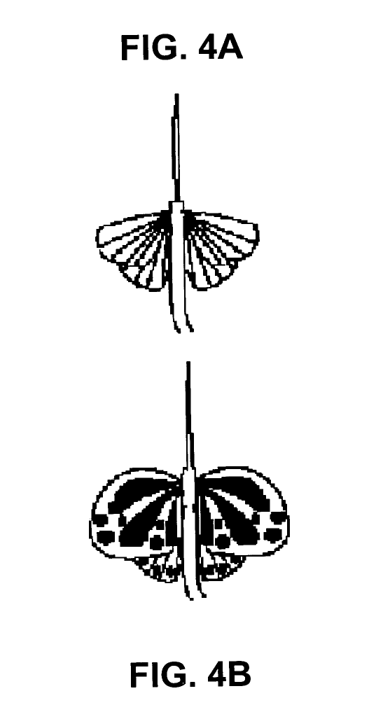

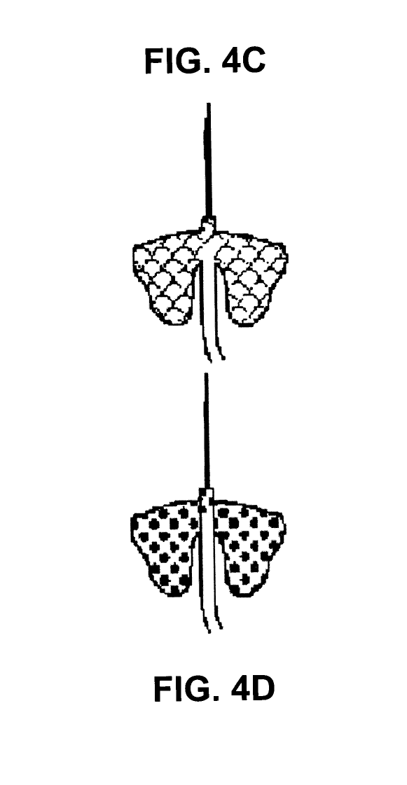

Wings of the present invention may also be covered with interesting and colorful geometric and design patterns as shown in FIGS. 4A, 4B, 4C and 4D. It is contemplated by the present invention that design artists may further this concept by making their own artistic designs for these wings in terms of shape, color, and design. These changes in color, design, and pattern may be integrated into the plastic or composition of the wing, or may be painted, printed, extruded, pasted, bonded, or otherwise fixed onto the surface of the wings, needle assembly, and/or shield. FIGS. 2A, 2B, 2C and 2D illustrate possible alternatives in wing design and shape. FIGS. 3A, 3B, 3C and 3D illustrate alternative placements for wing attachment along the lumen of a needle or catheter, in this case, for example, representing a butterfly or moth.

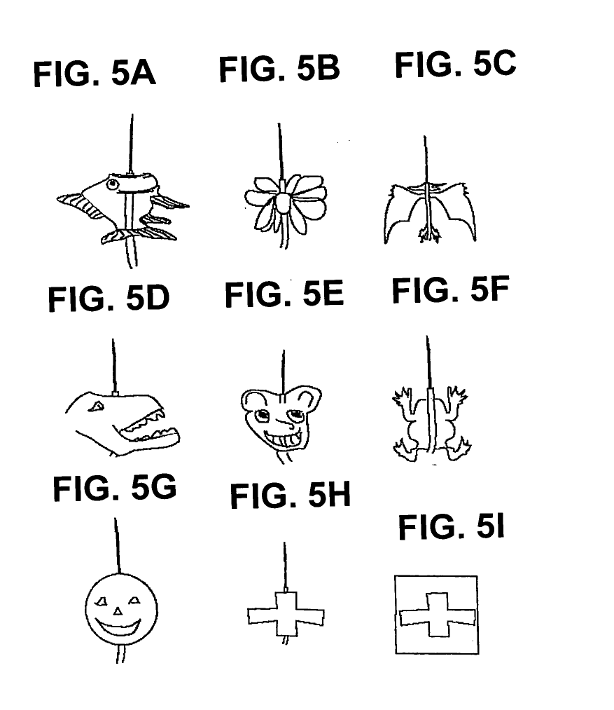

The wings or flat surfaces do not have to resemble butterflies, moths, or other insects or arthropods, but many other wing designs, fixing surfaces, and aesthetic and artistic changes are possible for butterfly needles and other catheters and medical devices with flat or nearly flat surfaces that may accommodate such designs. FIGS. 5A, 5B, 5C, 5D, 5E, 5F, 5G and 5H illustrate a few possible examples of non-butterfly shapes and designs for these butterfly needles. FIG. 5A demonstrates a fish, dolphin, whale or other sea animal; the orientation illustrated is just an example and may be rotated in any direction. FIG. 5B illustrates a flower or any other organic product such as leaves, fruits or vegetables. FIG. 5C suggests a winged reptile but may be any flying animal, imaginary or real. FIG. 5D demonstrates a dinosaur or dragon. FIG. 5E represents a cartoon character. The cartoon character may be a unique or a trademarked or copyrighted cartoon character of any sort, and all are contemplated by the present invention. FIG. 5F represents a four-legged animal but may be any amphibian, reptile, or mammal with various legs or appendages. FIG. 5G is a jack-o-lantern representative of any holiday symbol, FIG. 5H is a religious symbol suggested to offer serenity to the patient.

FIG. 5I illustrates a bandage designed to coordinate with the design of FIG. 5H. The adhesive patch, sticker or bandage of FIG. 5I may be purely ornamental or may be a functional bandage or dressing, and composed of a plastic or paper biocompatible surface, a biocompatible adhesive or foam adhesive on the skin side, and/or a peel away plastic or plasticized paper to expose the adhesive. This patch, sticker or bandage may come packaged with a needle so that if the needle device were covered with an opaque tape, bandage or dressing and the colorful butterfly needle could not be seen, the patient may be pleased and reminded by the adhesive patch with the same design of the underlying novel catheter or medical device. All of the above-identified classes of designs and colors for the colorful butterfly needle are applicable to the patches, stickers and bandages as well.

After a catheter, needle or butterfly needle is inserted into a vein, the apparatus must be stabilized, or it may twist and rip out of the vein. The initial step to stabilize a butterfly needle after insertion of the needle or catheter into a vein requires folding down the wings onto skin and fixing them onto the skin with medical adhesive tape. However, there are moments of instability while the operator is holding down the butterfly needle with one hand, and attempting to find a piece of tape with the other. In this moment, the butterfly needle may become dislodged, abrogating the entire procedure. Thus, an innovation to easily fix butterfly wings to skin and permit more controlled taping or fixation would also be useful.

U.S. Pat. No. 3,885,560 to Baldwin approaches the fixation difficulties by having an entire needle apparatus covered with a folded bandage that may be extended after the needle is inserted. After use, the butterfly needle may be removed and the bandage may remain to dress the wound. This is not truly a method of fixing, but rather a dressing, completely surrounding the butterfly needle, and is rather bulky. U.S. Pat. No. 4,698,057 to Joishy discloses suction cups and rolls of tape on the wings. However, it is difficult to unroll the rolls of tape.

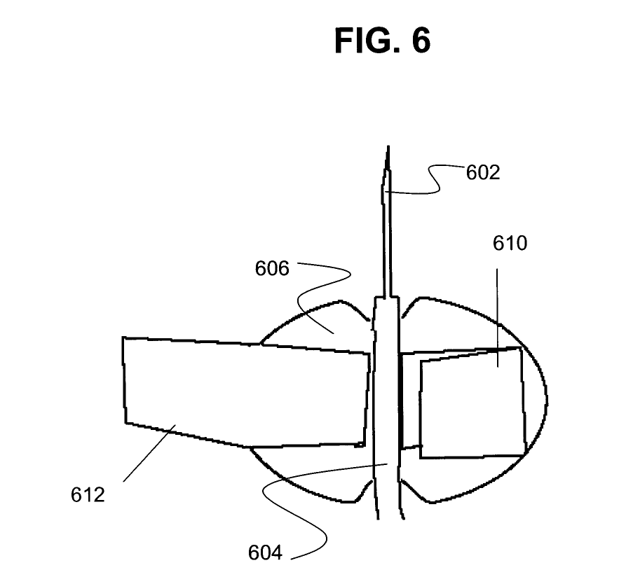

The present invention approaches the fixation difficulties in a different manner. FIG. 6 is an example of a dorsal-based securing system comprised of folded adhesive tape 610 which forms flaps 612 to be extended laterally, forward, or backward to secure the wings 606 of the catheter 604 with needle 602. When the wings 606 are folded for insertion of the catheter, these flaps remain folded between the two wings, so that insertion is identical to a conventional butterfly needle or butterfly shield.

Another solution to address the fixation difficulties is the addition of an adhesive to the skin-side surface of the butterfly wings. U.S. Pat. No. 4,324,236 to Gordon discloses a set of adhesive wings and a set of non-adhesive wings on the same catheter. This has obvious disadvantages of complexity and redundancy. U.S. Pat. No. 4,627,842 to Katz discloses the placement of adhesive on the wings of a conventional butterfly needle. While the Katz system rapidly anchors the needle, it interferes with removal of the needle in a conventional butterfly and inactivation of the needle when the needle must move into a shielded device for a shielded butterfly needle. In addition, when the adhesive covers are removed, the adhesive on the wings sticks not only to the patient's skin, but also the operator's fingers, thus, the needle becomes unstable as the operator attempts to fold down the wings and free his own fingers from the adhesive. U.S. Pat. No. 5,178,157 to Fanlo devises adhesive on the wings, but the wings are stilted to hold the position of the catheter at an angle, not taped flush with the skin. U.S. Pat. No. 5,704,917 to Utterberg applies adhesive to the shield, which in turn, fixes the shield to the skin, so that the conventional butterfly needle may be retracted into the shield and the shield may remain fixed to the skin. The main disadvantage to this arrangement is that the surface area of the shield is limited such that pulling on the catheter may break the adhesive bond.

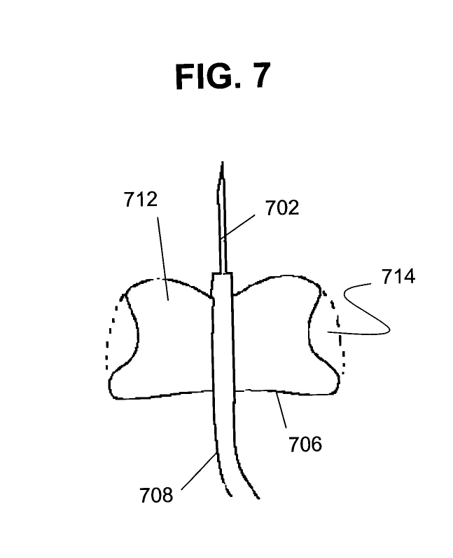

The present invention approaches fixation of the wings with adhesive on the skin surface of the wings in two examples: 1) a traditional butterfly needle without a shield, and 2) a butterfly needle shield wherein the shield has wings, but the needle assembly does not have wings. FIG. 7 illustrates, in a view from the underside of the butterfly needle assembly, an embodiment wherein adhesive or adhesive foam 712 covers a portion of the wing bottoms, except for a finger-gripping area 714. This non-adhesive grip allows the fingers to be free of adhesive and therefore the wings 706 may be easily handled while the needle 702 is pushed into a patient's vein. Fluid flows to needle 702 through tubing 708. The finger-gripping area 714 may be textured or ridged to prevent slippage.

The danger from hypodermic needles has also been reduced by the design of a new family of shielded butterfly needles and catheters. This family of shielded butterfly needles may be inactivated with one hand, unlike conventional butterfly needles that require two hands. This requires special and unique modifications of the shield and needle to permit the index finger to rest on a tab or grip that moves the needle into the shield using a dorsal slot or equivalent. A thumb rest may be added to the shield to permit the thumb to provide the force necessary to move the needle into the shield by providing an opposing force in the direction of the index finger in a “pinch” movement. The thumb rest also permits the tubing to move freely out of the shield as the thumb is depressed, unlike any conventional shielded butterfly needle. The needles are best inactivated while they are still taped to the skin using the one-handed technique.

U.S. Pat. No. 6,379,335 to Rignon et al., U.S. Pat. No. 5,350,368 to Shields, and U.S. Pat. No. 5,921,969 to Vallelunga et al., disclose different shielding solutions such as a sleeve or pocket into which the butterfly needle is retracted. A disadvantage of these systems is that the needle is pulled into the sleeve or pocket by the catheter, requiring two hands, and permitting the needle to shift dangerously. These pockets are also rather bulky and subject to contamination since the fabric may hold debris, bacteria, and fluids. Additional prior art including U.S. Pat. No. 5,030,212 to Ryan, U.S. Pat. No. 5,951,525 to Thorne et al., and U.S. Pat. No. 6,001,083 to Wilner, similarly struggle with single-handed inactivation.

Another shielding solution places the wings on the shield, rather than the needle assembly, and the needle may be pulled into the shield by the tubing as disclosed in U.S. Pat. No. 4,969,876 to Patterson and U.S. Pat. No. 5,088,982 to Ryan. To inactivate either of these devices, the needle assembly must be unlocked from the shield, and then the device may be pulled into the shield. Again, the shield is generally held with one hand as the needle is inactivated by another hand.

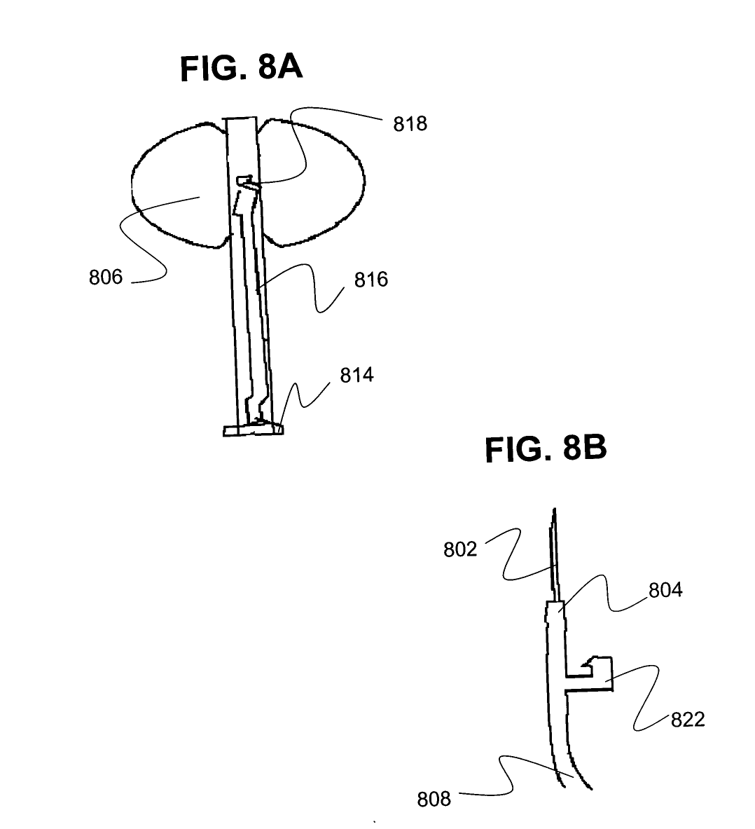

The present invention permits one-handed inactivation of a winged needle system with wings on the shield. FIGS. 8A and 8B illustrate a shielded needle 802 of catheter 804 with wings 806 on the shield, as well as a locking device to stabilize the needle while being inserted into a patient. A dorsal track 816 in the shield monitors movement of the actuator along the hilt of the shield. The locking device can be part of the shield when locked to the actuator or incorporated into the track trapping the actuator. Alternatively, the locking device may comprise a tab 822 to interlock with a recess 818 of a different angle or shape, or may be on the needle assembly, tubing 808, and shield such that the needle assembly or tubing locks into the shield.

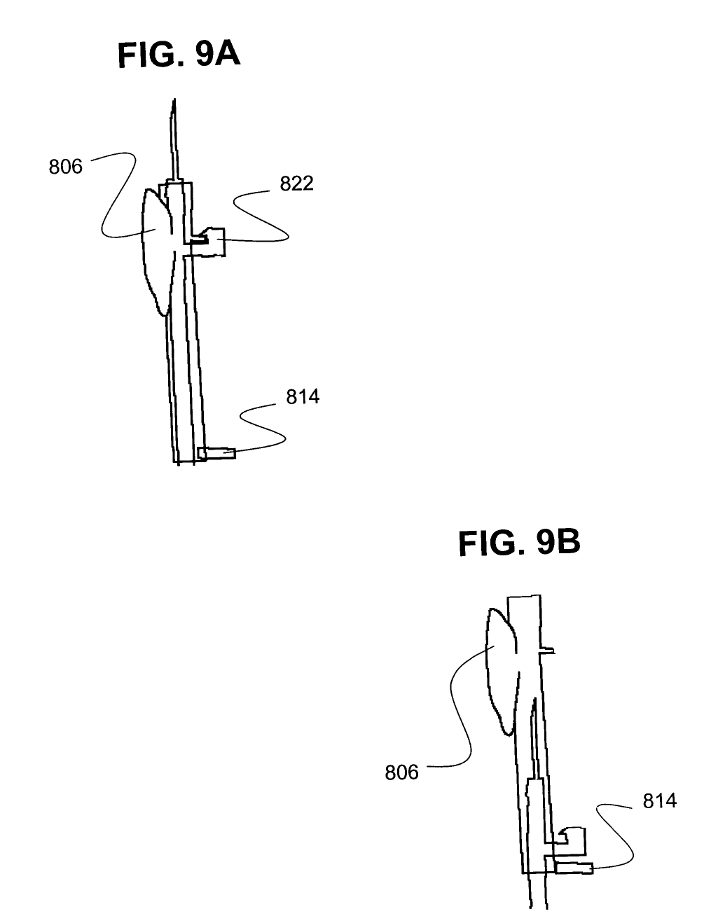

FIGS. 9A and 9B illustrate an assembled butterfly needle. FIG. 9A presents a side view of a butterfly shield with the needle assembly in the extended position. To shield the needle assembly, the locking device or tab of the needle assembly may be disengaged with the index finger of one hand, while the thumb of that same hand is placed on the thumb rest 814. The thumb rest 814 is above the catheter 804 such that the catheter may move out of the shield unimpeded by the thumb. This feature of one-hand inactivation is different than any other shielded winged needle or catheter system. A thumb placed on the end of the shield impedes the catheter outflow in systems without a thumb rest, thus preventing one-handed inactivation. In the present invention, the locking device or tab 822 of the needle assembly is moved by the index finger, along the dorsal track toward the thumb rest, and while the thumb remains on the thumb rest, joins the index finger in a “pinching motion”. The needle assembly is then retracted and locked into the shield. This one-handed inactivation works whether or not the shield remains fixed to the skin as long as the locking device or tab in the needle assembly can move freely in the dorsal track. This device may also be inactivated conventionally by fixing the shield and pulling on the catheter.

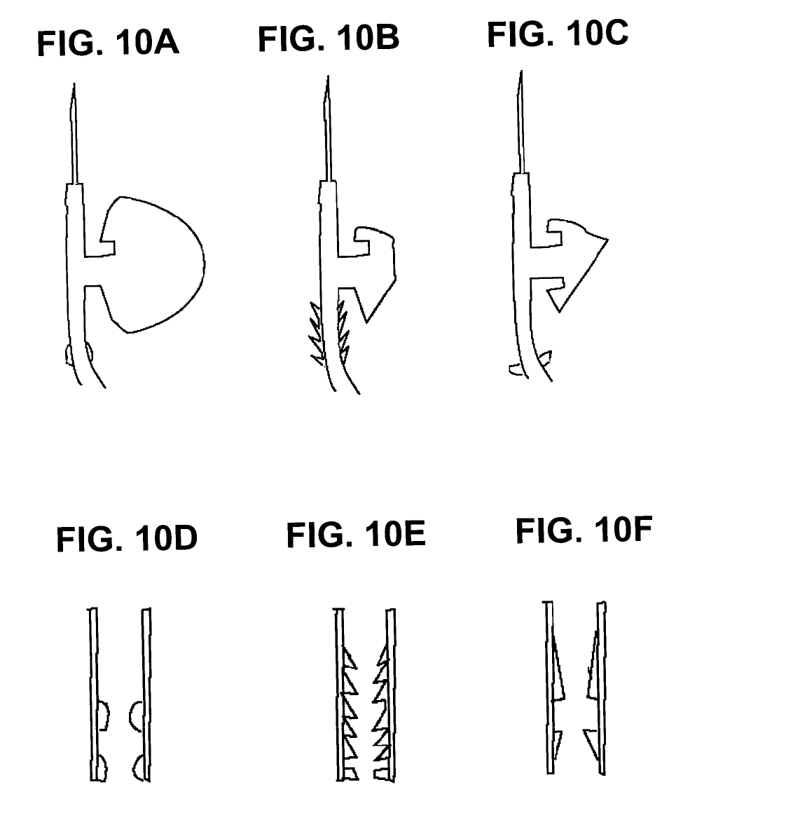

As previously discussed, the locking device may take various forms. FIGS. 10A, 10B and 10C suggest alternative locking devices for the needle assembly and shield. A locking device on the actuator or tab consisting of a slot that may accommodate a tab or projection from the forward section of the shield to fix the needle assembly in an extended position is shown. Alternatively, a third butterfly wing is folded down on the skin and taped like a conventional wing. A similar notch or slot on the locking device of the needle assembly, fitted with a finger release on the opposite side, may be pressed by the index finger to disengage the needle assembly from the tab or locking projection on the shield. A double locking device may have a notch or slot on each side, one for locking the needle assembly extended and the other for locking the needle assembly in the retracted position. This locking device is potentially reversible.

Devices to lock the needle assembly permanently in the retracted position are also possible, and examples of these are shown in FIGS. 10D, 10E and 10F. These involve locking devices on the needle assembly or tubing, as well as corresponding mating systems within the lumen of the shield. FIGS. 10D, 10E and 10F demonstrate embodiments of tapered and interlocking rings or tabs, oppositely directed and interlocking ratchet projections, and interlocking rings, ridges, or shaped projections trapped in a space created by a tapered dentate and surface, or two oppositely directed ratchet projections.

U.S. Pat. No. 5,279,588 to Nicoletti et al., U.S. Pat. No. 5,549,571 to Sak, U.S. Pat. 5,330,438 to Gollobin et al., U.S. Pat. No. 5,120,320 to Fayngold, and U.S. Pat. No. 5,704,917 to Utterberg all demonstrate the common shielded butterfly needles involving a standard butterfly needle within a specialized shield. These devices generally consistent of a largely conventional butterfly needle with wings, a shield with two side slots to accommodate movement of the wings, and a locking device. Virtually all of these systems require the needle to be inactivated by holding the shield and pulling on the catheter, and therefore none are inactivated with a single hand. When one attempts to inactivate these devices with one hand by pressing on the wing with the index finger the wings twist ineffectually and jam in the shield. When two fingers, the index and middle fingers, are used to move the wings and needle assembly, the tubing bunches up against the thumb because there is no thumb rest.

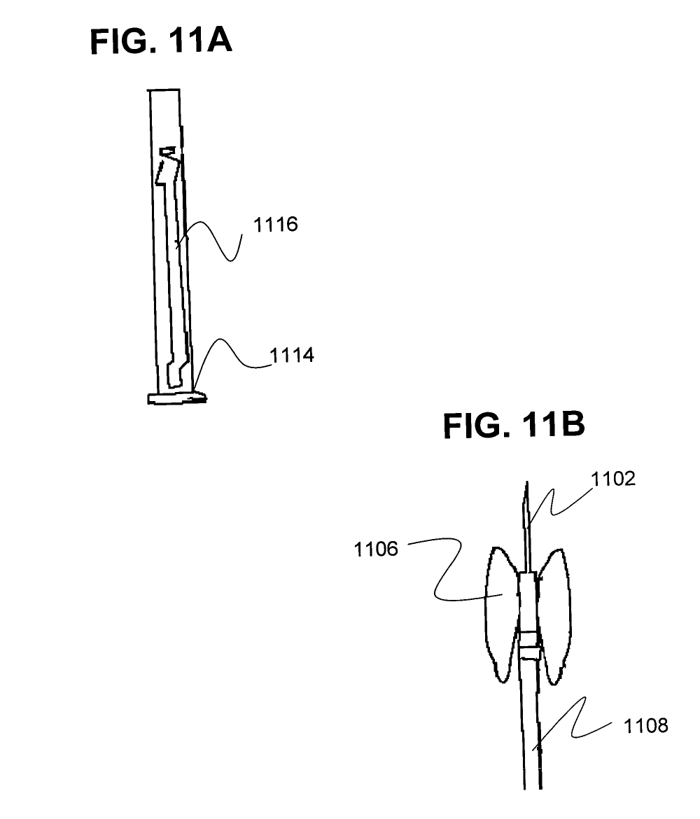

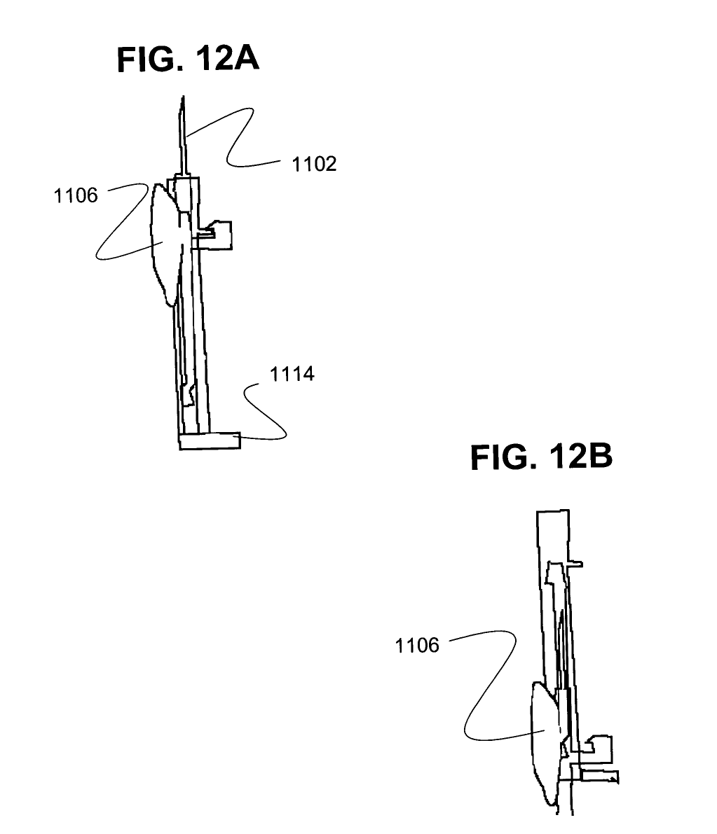

FIGS. 11A and 11B illustrate the components of a butterfly needle with the wings 1106 attached to the hub of the needle 1102 to pass along tracks 1116 in the shield. Similar to the embodiment in which the wings attach to the shield, as shown in FIGS. 8A and 8B, the embodiment of FIGS. 11A and 11B contains a locking device in the shield and a thumb rest 1114 above the plane of the shield so that the tubing 1108 may move out of the shield while the thumb is on the thumb rest. FIGS. 12A and 12B show an assembled winged needle 1102 movable within the shield. As illustrated, the thumb rest 1114 is above the catheter such that the catheter may move out of the shield unimpeded by the thumb. This arrangement, the thumb rest and one-hand inactivation, is not found with any other shielded winged needle or catheter system. In systems without a thumb rest, the thumb at the end of the shield blocks catheter outflow and prevents one-handed inactivation. In the present invention, the locking device or tab of the needle assembly may be moved by the index finger along the dorsal track toward the thumb rest, while the thumb remains on the thumb rest, with the two fingers (the index finger and thumb) coming together in a “pinching motion”. The needle assembly may then be retracted and locked into the shield. Thus, the present invention provides a shielded needle assembly designed to be inactivated with one hand. This one-handed inactivation works when the shield remains fixed to the skin by adhesive so long as the locking device and wings of the needle assembly can move freely in the dorsal and side tracks. The device of the present invention may also be conventionally inactivated by fixing the shield in place and pulling on the catheter.

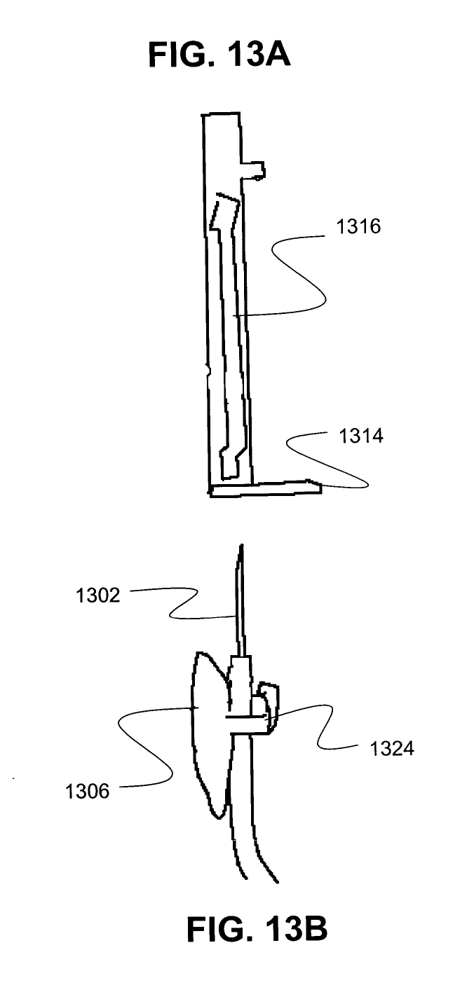

A shielded butterfly needle that may be inactivated with one hand as described above, may also be accomplished with a winged needle assembly and shield with only the side slits or tracks and not the dorsal slits or tracks. The components of this device, the arch shielded butterfly needle, are shown in FIGS. 13A and 13B. This consists of a shield, a locking device on the shield side tracks 1316 for movement of wing 1306 and a thumb rest 1314. A ring or arch 1324 attached to the wings 1306 or needle assembly encompasses the upper portion of the shield. The arch may also be moved forward beyond the wings on the needle assembly, so that the wings may be folded and not entrap the arch.

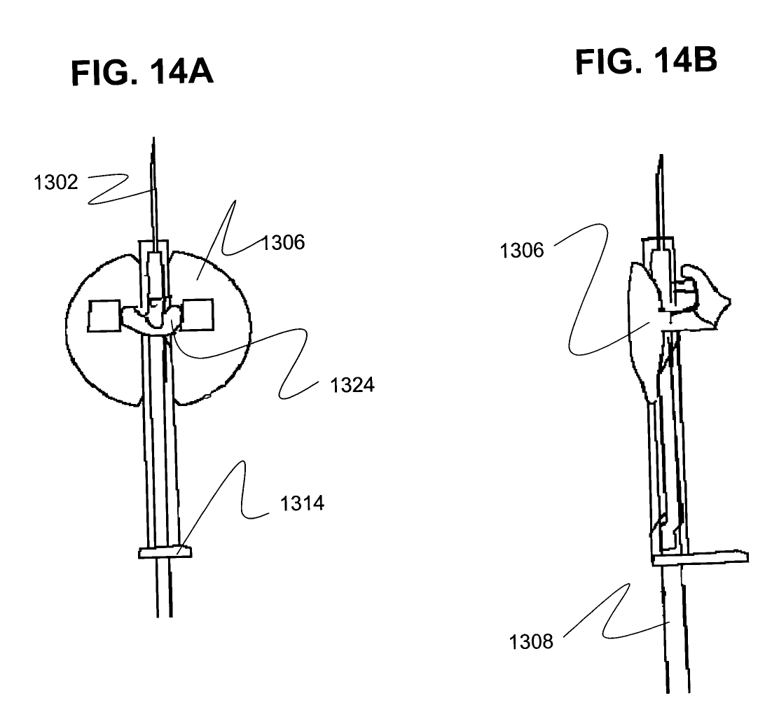

FIGS. 14A and 14B show an assembled arch shielded butterfly needle. The shielded butterfly needle of FIGS. 14A and 14B may be moved and inactivated identically to the above-described embodiments with all the same advantages, and the ability to inactivate the needle assembly with one hand by the same technique. This device may also be inactivated conventionally by fixing the shield to the skin and pulling on the catheter.

One-handed shielding of the butterfly needle, or any needle or catheter system, has also been improved with the addition of a reciprocating mechanism. This mechanism, which may be either line, gear, or hydraulic driven, connects the needle apparatus with a plunger or tab in a track. Thus, when the plunger or tab is moved forward in the track, the needle is retracted into the shield by this mechanism. The most favorable version of which is a line or filament pulley system that connects the plunger or tab to the needle, using the housing of the shield as a pulley, or alternatively by using another low friction device such as a conventional wheel-like pulley as the pulley device. This device may be easily operated with a single hand while maintaining absolute control of the needle and shield. These needles may also be inactivated similarly to other conventional shielded butterfly needles, by holding the shield and pulling on the tubing. This is similar to that used for the reciprocating syringe as disclosed in U.S. Pat. No. 6,245,046, the entire contents and disclosure of which is hereby incorporated by reference.

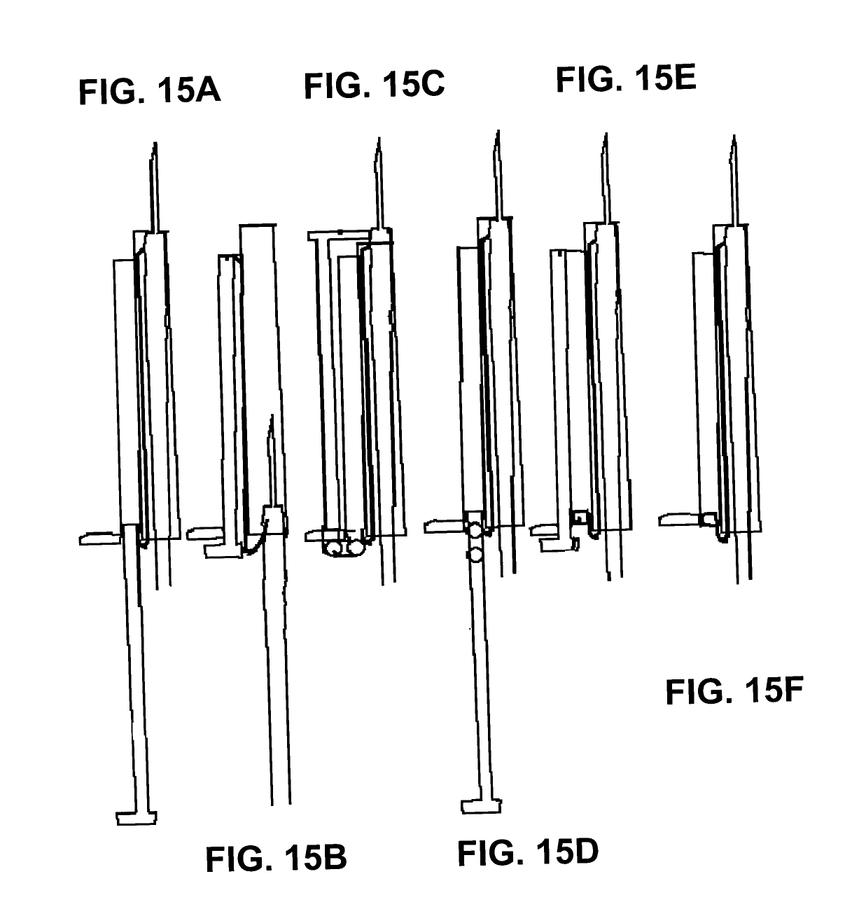

FIGS. 15A, 15B, 15C, 15D, 15E and 15F illustrate various assembled embodiments of a reciprocating needle. FIG. 15A demonstrates an assembled device with the plunger extended, and the driveline attached to the plunger and to the needle assembly. FIG. 15B shows the plunger depressed, the needle retracted and inactivated. It is contemplated by the present invention that the locking devices may include all of those delineated in the above-discussed embodiments. FIG. 15C shows an embodiment with a hinge in the plunger to permit it to be folded forward. The thumb rest of the plunger could be used as a device to lock the needle assembly in place during needle insertion. FIG. 15D demonstrates the plunger unfolded and extended and the needle unlocked, ready for inactivation. FIG. 15E shows a removable plunger, storable in a holder on the shield, which may also be used to lock and unlock the needle assembly. In this embodiment, the plunger pushes the line driver in the track. FIG. 15F illustrates an embodiment wherein the line driver is extended, this extension being movable in a track whereby the extension may be used as a tab, handle or driver to propel the line driver forward and inactivate the needle.

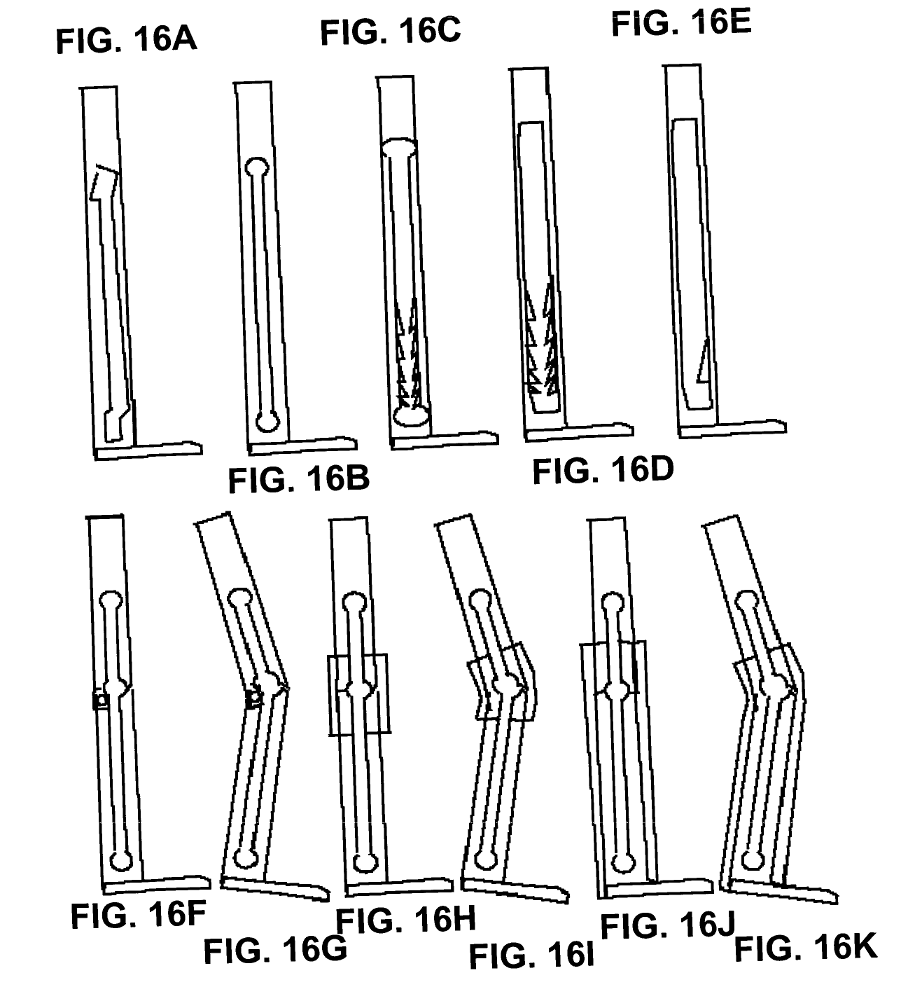

The wing tracks or slots as well as the dorsal tracks, which have been described above in multiple embodiments, may be of alternative design, some of which may serve as locking devices. The simplest form is a slit or track free of a locking formation. FIGS. 16A, 16B, 16C, 16D and 16E are examples of slot variants and slot locking mechanisms. FIG. 16A has an angled rectangle at each end to trap the rectangular shape of a wing attachment on the lateral tracks or the drive tab of the dorsal slot. FIG. 16B has a curvilinear void at each end of the track to capture an attachment. FIG. 16C is similar to FIG. 16B but for an irreversible locking system composed of unidirectional dentates. FIG. 16D is of the same design concept, but the track is a simple rectangular track. FIG. 16E has a single dentate which would irreversibly lock a number of attachment designs.

Another major problem with many shielded needle devices, especially those with a rigid shield, is that the shield makes the butterfly needle device effectively longer thereby creating a longer lever arm. With a longer effective device, slight changes in orientation may cause major changes in the position of the needle tip in relation to the fulcrum of the device causing disruption of the blood vessel or painful tension on the tissues. This longer lever arm becomes especially evident when the device is taped to the skin or manipulated. Thus, a solution to prevent the deleterious effects of the longer lever arm caused by the shield may also be a major advance in the stability of these needles.

In the present invention, the mechanical disadvantage induced by the longer lever arm has been reduced by the addition of a restricted hinge in the shield, the addition of a flexible shield, or the addition of a flexible shield segment. All of these modifications reduce the effective lever arm to alleviate the negative mechanical aspects of a butterfly or needle shield. FIGS. 16F, 16G, 16H, 16I, 16J and 16K illustrate flexible shields in extended and flexed positions. FIGS. 16F and 16G demonstrate a rigid shield with a hinge, in the flexed and extended positions. FIGS. 16H and 16I show a rigid shield with a flexible joint that functions as a hinge equivalent. The lateral track for the wing extends through this flexible segment. FIGS. 16J and 16K demonstrate a rigid fore-section and a flexible portion to permit the appropriate movement and decreasing the effective lever arm. An additional variant may comprise an entire flexible shield, flexible side-to-side to some degree, but not compressible significantly axially, so that the retracted needle would not be exposed. All of these designs may incorporate a stop to limit motion, or may have limitations in flexibility, because if the shield may flex completely, the reversed needle may stick the operator.

All documents, patents, journal articles and other materials cited in the present application are hereby incorporated by reference.

Although the present invention has been fully described in conjunction with the preferred embodiment thereof with reference to the accompanying drawings, it is to be understood that various changes and modifications may be apparent to those skilled in the art. Such changes and modifications are to be understood as included within the scope of the present invention as defined by the appended claims, unless they depart therefrom.

1. A butterfly needle assembly comprising:
a needle having a needle hub;
a locking means integral with said needle hub having a tab protruding radially from said needle hub; and
a shield with a distal end and a proximal end having integral wings and a dorsal track extending axially along said shield;
wherein said tab extends through said dorsal track such that when in operation as said needle moves through said shield, the tab travels along the dorsal track to engage a cut-out at the distal end of said dorsal track thereby locking the needle within said shield.

2. The butterfly needle assembly of claim 1, wherein said shield has a thumb rest on said proximal end.

3. The butterfly needle assembly of claim 1, wherein said integral wings resemble wings of a butterfly.

4. The butterfly needle assembly of claim 1, wherein said integral wings resemble wings of a moth.

5. The butterfly needle assembly of claim 1, wherein said integral wings resemble an animal.

6. The butterfly needle assembly of claim 1, wherein said integral wings resemble a flower.

7. The butterfly needle assembly of claim 1, wherein said integral wings resemble a prehistoric creature.

8. The butterfly needle assembly of claim 1, wherein said integral wings resemble a cartoon character.

9. The butterfly needle assembly of claim 1, wherein said integral wings resemble a holiday novelty.

10. The butterfly needle assembly of claim 1, wherein said integral wings have folded adhesive tabs on the underside of said integral wings.

11. The butterfly needle assembly of claim 1, wherein said integral wings have adhesive tape on the underside of said integral wings.

12. The butterfly needle assembly of claim 1, wherein a mechanism facilitates reciprocal movement of said needle with said shield.

13. The butterfly needle assembly of claim 1, wherein said needle irreversibly locks within said shield.

14. The butterfly needle assembly of claim 1, wherein said shield has a flexible portion axially displaced from both said proximal and said distal end.

15. The butterfly needle assembly of claim 2, wherein said integral wings resemble wings of a butterfly.

16. The butterfly needle assembly of claim 2, wherein said integral wings resemble wings of a moth.

17. The butterfly needle assembly of claim 2, wherein said integral wings resemble an animal.

18. The butterfly needle assembly of claim 2, wherein said integral wings resemble a flower.

19. The butterfly needle assembly of claim 2, wherein said integral wings resemble a prehistoric creature.

20. The butterfly needle assembly of claim 2, wherein said integral wings resemble a cartoon character.

21. The butterfly needle assembly of claim 2, wherein said integral wings resemble a holiday novelty.

22. The butterfly needle assembly of claim 2, wherein said integral wings have folded adhesive tabs on the underside of said integral wings.

23. The butterfly needle assembly of claim 2, wherein said integral wings have adhesive tape on the underside of said integral wings.

24. The butterfly needle assembly of claim 2, wherein a mechanism facilitates reciprocal movement of said needle with said shield.

25. The butterfly needle assembly of claim 2, wherein said needle irreversibly locks within said shield.

26. The butterfly needle assembly of claim 2, wherein said shield has a flexible portion axially displaced from both said proximal and said distal end.

27. A butterfly needle comprising:
a needle having a needle hub;
wherein said needle hub has a pair of wings extending therefrom and said wings are aesthetically decorated.

28. The butterfly needle of claim 27, wherein said wings resemble wings of a butterfly.

29. The butterfly needle of claim 27, wherein said wings resemble wings of a moth.

30. The butterfly needle of claim 27, wherein said wings resemble an animal.

31. The butterfly needle of claim 27, wherein said wings resemble a flower.

32. The butterfly needle of claim 27, wherein said wings resemble a prehistoric creature.

33. The butterfly needle of claim 27, wherein said wings resemble a cartoon character.

34. The butterfly needle of claim 27, wherein said wings resemble a holiday novelty.

35. A butterfly needle assembly comprising:
a needle having a needle hub with integral wings extending radially from said needle hub;
a locking means integral with said needle hub having a tab protruding radially from said needle hub in a plane perpendicular to said integral wings; and
a shield with a distal end and a proximal end and a dorsal track extending axially along said shield and said shield further having side tracks;
wherein said tab extends through said dorsal track and said integral wings extend through said side tracks such that when in operation as said needle moves through said shield, the tab travels along the dorsal track to engage a cut-out at the distal end of said dorsal track thereby locking the needle within said shield.

36. The butterfly needle assembly of claim 35, wherein said shield has a thumb rest on said proximal end.

37. The butterfly needle assembly of claim 35, wherein said integral wings resemble wings of a butterfly.

38. The butterfly needle assembly of claim 35, wherein said integral wings resemble wings of a moth.

39. The butterfly needle assembly of claim 35, wherein said integral wings resemble an animal.

40. The butterfly needle assembly of claim 35, wherein said integral wings resemble a flower.

41. The butterfly needle assembly of claim 35, wherein said integral wings resemble a prehistoric creature.

42. The butterfly needle assembly of claim 35, wherein said integral wings resemble a cartoon character.

43. The butterfly needle assembly of claim 35, wherein said integral wings resemble a holiday novelty.

44. The butterfly needle assembly of claim 35, wherein said integral wings have folded adhesive tabs on the underside of said integral wings.

45. The butterfly needle assembly of claim 35, wherein said integral wings have adhesive tape on the underside of said integral wings.

46. The butterfly needle assembly of claim 35, wherein a mechanism facilitates reciprocal movement of said needle with said shield.

47. The butterfly needle assembly of claim 35, wherein said needle irreversibly locks within said shield.

48. The butterfly needle assembly of claim 35, wherein said shield has a flexible portion axially displaced from both said proximal and said distal end.

49. The butterfly needle assembly of claim 36, wherein said integral wings resemble wings of a butterfly.

50. The butterfly needle assembly of claim 36, wherein said integral wings resemble wings of a moth.

51. The butterfly needle assembly of claim 36, wherein said integral wings resemble an animal.

52. The butterfly needle assembly of claim 36, wherein said integral wings resemble a flower.

53. The butterfly needle assembly of claim 36, wherein said integral wings resemble a prehistoric creature.

54. The butterfly needle assembly of claim 36, wherein said integral wings resemble a cartoon character.

55. The butterfly needle assembly of claim 36, wherein said integral wings resemble a holiday novelty.

56. The butterfly needle assembly of claim 36, wherein said integral wings have folded adhesive tabs on the underside of said integral wings.

57. The butterfly needle assembly of claim 36, wherein said integral wings have adhesive tape on the underside of said integral wings.

58. The butterfly needle assembly of claim 36, wherein a mechanism facilitates reciprocal movement of said needle with said shield.

59. The butterfly needle assembly of claim 36, wherein said needle irreversibly locks within said shield.

60. The butterfly needle assembly of claim 36, wherein said shield has a flexible portion axially displaced from both said proximal and said distal end.
